(19) United States
(12) Patent Application Publication
(10) Pub. No.: US 2021/0383917 A1
(43) Pub. Date: Dec. 9, 2021

(54) NURSING ASSISTANCE DEVICE, NURSING ASSISTANCE METHOD, AND RECORDING MEDIUM

(71) Applicant: NEC Corporation, Minato-ku, Tokyo (JP)

(72) Inventors: Yuki KOSAKA, Tokyo (JP); Masahiro KUBO, Tokyo (JP); Yuan LUO, Tokyo (JP); Shigemi KITAHARA, Tokyo (JP)

(73) Assignee: NEC Corporation, Minato-ku, Tokyo (JP)

(21) Appl. No.: 17/285,230

(22) PCT Filed: Oct. 15, 2019
(86) PCT No.: PCT/JP2019/040443

§ 371 Date: Apr. 14, 2021

(30) Foreign Application Priority Data
Oct. 23, 2018 (JP) 2018-199529

(51) Int. Cl. G16H 40/20; G06F 40/268; G06F 40/279; G16H 10/60

(52) U.S. Cl. CPC G16H 40/20; G16H 10/60; G06F 40/279; G06F 40/268

(57) ABSTRACT

A nursing assistance device includes: a record omission determination unit that determines information whose record is omitted among nursing information related to nursing of a patient, based on a record omission determination condition related to the nursing information; and an output unit that outputs an input request for the determined information whose recording is omitted.

### TECHNICAL FIELD

The present invention relates to a nursing assistance device, a nursing assistance method, and a recording medium.

### BACKGROUND ART

Work of recording nursing information is very important in a nursing work for a patient, and important matters related to nursing need to be recorded without omission. Here, nursing information refers to general information that should be understood by a nurse in nursing a patient such as a condition of the patient, nursing content for the patient, and a change in the condition of the patient in the nursing work for the patient. A plurality of nurses involved in caring for a patient can understand the condition of the patient and can perform appropriate care for the patient based on the understanding by confirming the nursing information.

Here, technologies related to nursing information are disclosed in Patent Documents 1 to 3.

Patent Document 1 discloses a technology capable of improving the convenience of an electronic medical record by sufficiently ensuring accessibility of the electronic medical record and memo information.

Patent Document 2 discloses a technology for improving the quality of medical care information by preventing record omission and record errors in diagnostic information including a hygienist business record book, a periodontal inspection record table, and the like in a cross-sectoral manner.

Patent Document 3 discloses a technology for automatically showing problems and treatments of a patient or relatives of the patient in the past in the medical care of the patient in an electronic medical record and performing appropriate medical treatment and prescription for a highly hereditary disease.

### PRIOR ART DOCUMENTS

### Patent Document

### [Patent Document 1] Japanese Unexamined Patent Application, First Publication No. 2017-16216

### [Patent Document 2] Japanese Unexamined Patent Application, First Publication No. 2016-76190

### [Patent Document 3] Japanese Unexamined Patent Application, First Publication No. 2014-219714

### SUMMARY OF THE INVENTION

### Problem to be Solved by the Invention

In the above-mentioned technologies of the nursing information, for example, there is a need for technology that can prevent the omission of the record by appropriately pointing out that there is an omission in the content of the nursing information and prompting to input the nursing information and thereby enhances medical safety and reduces the labor of confirmation by a nurse or the like due to the record omission.

An example object of the present invention is to provide a nursing assistance device, a nursing assistance method, and a recording medium that solve the above-mentioned problems.

### Means for Solving the Problem

According to a first example aspect of the present invention, a nursing assistance device includes: a record omission determination unit that determines information whose record is omitted among nursing information related to nursing of a patient, based on a record omission determination condition related to the nursing information; and an output unit that outputs an input request for the determined information whose recording is omitted.

According to a second example aspect of the present invention, a nursing assistance method includes: determining information whose record is omitted among nursing information related to nursing of a patient, based on a record omission determination condition related to the nursing information; and outputting an input request for the determined information whose recording is omitted.

According to a third example aspect of the present invention, a recording medium stores a program for causing a computer to execute: determining information whose record is omitted among nursing information related to nursing of a patient, based on a record omission determination condition related to the nursing information; and outputting an input request for the determined information whose recording is omitted.

### Effect of the Invention

According to an example embodiment of the present invention, it is possible to provide a nursing assistance device that enhances medical safety by preventing the omission of a record and reduces the labor of confirmation by a nurse or the like due to the record omission.

### BRIEF DESCRIPTION OF THE DRAWINGS

FIG. 1 is a diagram showing an outline of a nursing assistance system according to an example embodiment of the present invention.

FIG. 2 is a diagram showing a hardware configuration of a nursing assistance device according to the example embodiment of the present invention.

FIG. 3 is a functional block diagram of the nursing assistance device according to the example embodiment of the present invention.

FIG. 4 is a first diagram showing a data example stored in the nursing assistance device according to the example embodiment of the present invention.

FIG. 5 is a second diagram showing the data example stored in the nursing assistance device according to the example embodiment of the present invention.

FIG. 6 is a third diagram showing the data example stored in the nursing assistance device according to the example embodiment of the present invention.

FIG. 7 is a fourth diagram showing a data example stored in the nursing assistance device according to the example embodiment of the present invention.

FIG. 8 is a first diagram showing a processing flow of the nursing assistance device according to the example embodiment of the present invention.

FIG. 9 is a second diagram showing the processing flow of the nursing assistance device according to the example embodiment of the present invention.

FIG. 10 is a diagram showing a first example of a UI screen according to the example embodiment of the present invention.

FIG. 11 is a diagram showing a second example of the UI screen according to the example embodiment of the present invention.

FIG. 12 is a diagram showing a nursing assistance device according to another example embodiment of the present invention.

### EXAMPLE EMBODIMENT

Hereinafter, a nursing assistance device 1 according to an example embodiment of the present invention will be described with reference to the drawings.

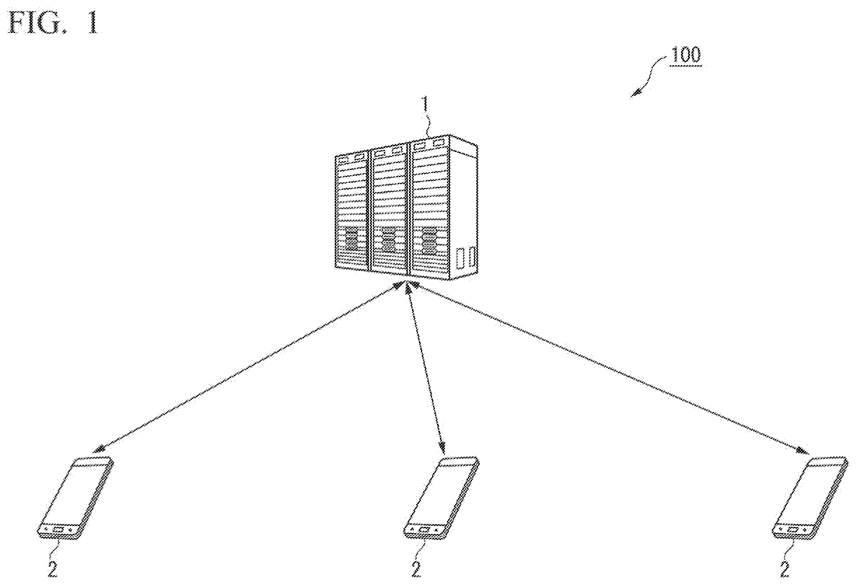

FIG. 1 shows an outline of a nursing assistance system 100 having the nursing assistance device 1 according to the present example embodiment.

The nursing assistance device 1 is installed in a hospital or the like. The nursing assistance device 1 may be installed outside the hospital. The nursing assistance device 1 is communicably connected to terminals 2 such as smartphones. The nursing assistance system 100 is constituted by the nursing assistance device 1 and the terminals 2. The terminal 2 is owned by, for example, a nurse or a doctor. The terminal 2 may be a PC or the like instead of a mobile terminal such as a smartphone.

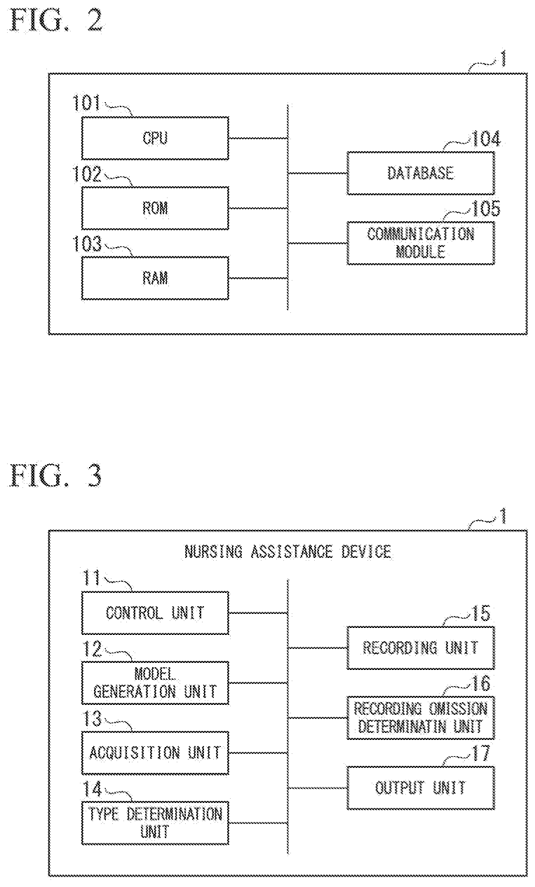

FIG. 2 is a diagram showing a hardware configuration of the nursing assistance device 1.

As shown in FIG. 2, the nursing assistance device 1 is a computer that includes hardware such as a central processing unit (CPU) 101, a read only memory (ROM) 102, a random access memory (RAM) 103, a database 104, and a communication module 105.

FIG. 3 is a functional block diagram of the nursing assistance device 1.

The nursing assistance device 1 executes a nursing assistance program. Accordingly, the nursing assistance device 1 has functions of a control unit 11, a model generation unit 12, an acquisition unit 13, a type determination unit 14, a recording unit 15, a record omission determination unit 16, and an output unit 17.

The control unit 11 controls the other functional units.

The model generation unit 12 generates a record type determination model that specifies a record type for each nursing type based on pieces of past nursing information of a plurality of patients of which nursing types match each other. The record type determination model indicates any of a content determination model and a SOAP determination model. Details of the content determination model and the SOAP determination model will be described later.

The acquisition unit 13 acquires input information received from the terminal 2.

The type determination unit 14 determines at least a record type of the input information received from the terminal 2.

The recording unit 15 records, as nursing information, the input information associated with at least the record type.

The record omission determination unit 16 determines the nursing information whose record is omitted for the patient among the pieces of nursing information of the patients based on a record omission determination condition of the nursing information.

The output unit 17 outputs an input request for the nursing information whose record is omitted to the terminal 2.

In the present example embodiment, the record type is roughly classified into a SOAP type and a record content type. SOAP is a word taken from the initials of “Subjective (subjective information)”, “Objective (objective information)”, “Assessment (assessment information)”, and “Plan (plan, treatment information)”, and indicates the types of these pieces of information. Hereinafter, the type of “Subjective” is referred to as S, the type of “Objective” is referred to as O, the type of “Assessment” is referred to as A, and the type of “Plan” is referred to as P. “Subjective” indicates information obtained directly from the patient. “Objective” indicates information obtained from a physical examination and an inspection by the nurse. “Assessment” indicates information of assessment by the nurse or the doctor based on “Subjective” and “Objective”. “Plan” indicates a plan or a treatment performed as a result of “Assessment”.

The nursing assistance device 1 according to the present example embodiment determines the nursing information whose record is omitted for the patient among the pieces of nursing information of the patients based on the record omission determination condition of the nursing information, and outputs the input request for the nursing information whose record is omitted to the terminal 2.

Through such processing, the nursing assistance device 1 provides a technology for reducing a labor of a nursing work.

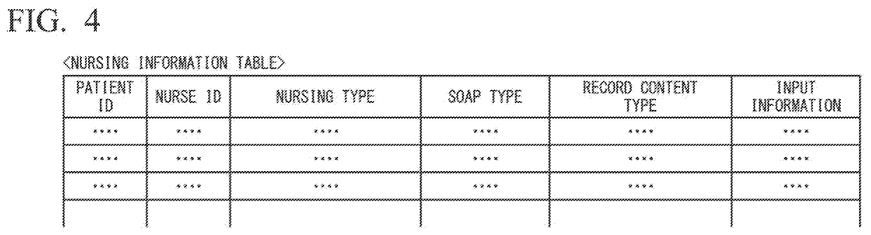

FIG. 4 is a first diagram showing a data example (nursing information table) stored in the nursing assistance device 1.

The nursing assistance device 1 records the nursing information associated with a patient ID, a nurse ID, a nursing type, a SOAP type, a record content type, input information, and the like in the nursing information table based on the acquisition of the input information received from the terminal 2. The nursing information table is recorded in the database 104. As an example, the nursing type indicates a type of a disease of the patient such as “fracture” or “stroke”. The nursing type may be information indicating a different type for each patient attribute such as a gender or an age group of the patient in addition to the type of the disease of the patient. The SOAP type indicates information for specifying that the input information is any type of “Subjective (subjective information)”, “Objective (objective information)”, “Assessment (assessment information)”, and “Plan (plan, treatment information)” described above. The record content type is information for specifying that the input information is any state of the patient. The input information is main information that should be recorded as the nursing information input by the user such as the nurse or the doctor via the terminal 2. The nursing information may not include the nursing type.

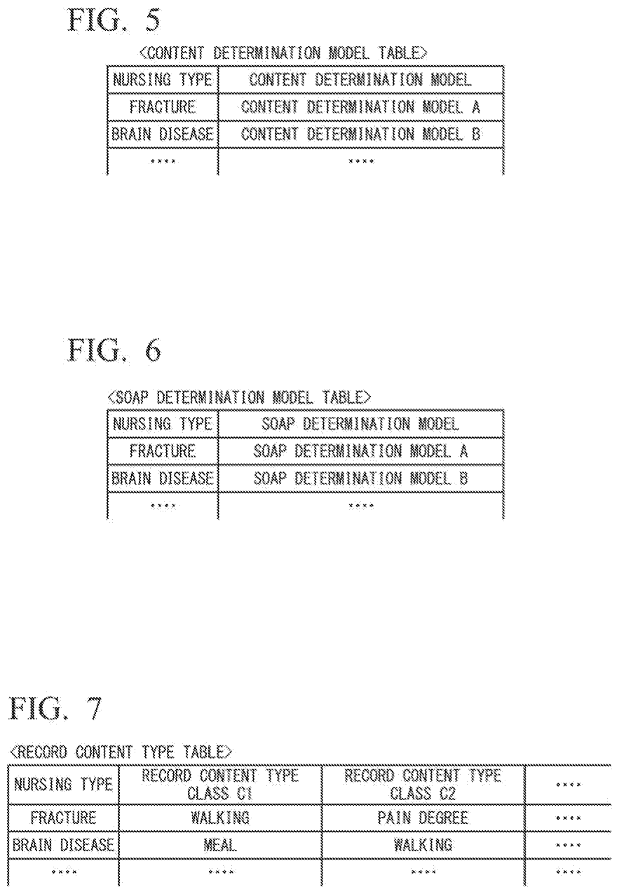

FIG. 5 is a second diagram showing a data example (content determination model table) stored in the nursing assistance device 1.

The content determination model is generated by the model generation unit 12 of the nursing assistance device 1 based on the pieces of past nursing information of the plurality of patients. The content determination model is provided for each nursing type. The content determination model is recorded in the database 104. The content determination model is a model for determining the record content type. The nursing assistance device 1 stores the content determination model table for specifying the content determination model corresponding to the nursing type in the database 104. The nursing assistance device 1 specifies the content determination model corresponding to the nursing type from this content determination model table, and determines the record content type of the input information by using the content determination model. In a case where the nursing type is not included in the nursing information, one content determination model may be recorded in the nursing assistance device 1, and the record content type of the input information may be determined by using this content determination model.

FIG. 6 is a third diagram showing a data example (SOAP determination model table) stored in the nursing assistance device 1.

The SOAP determination model is generated by the model generation unit 12 of the nursing assistance device 1 based on the pieces of past nursing information of the plurality of patients. The SOAP determination model is provided for each nursing type. The SOAP determination model is recorded in the database 104. The nursing assistance device 1 stores the SOAP determination model table for specifying the SOAP determination model corresponding to the nursing type. The nursing assistance device 1 specifies the SOAP determination model corresponding to the nursing type from this SOAP determination model table, and determines the SOAP type of the input information by using the SOAP determination model. In a case where the nursing type is not included in the nursing information, one SOAP determination model may be recorded in the nursing assistance device 1, and the record content type of the input information may be determined by using this SOAP determination model. For example, the nursing assistance device 1 determines which one of the record content types of “Subjective (subjective information)”, “Objective (objective information)”, “Assessment (assessment information)”, and “Plan (plan, treatment information)” the input information included in the nursing information is, by using the SOAP determination model.

FIG. 7 is a fourth diagram showing an example of data (record content type table) stored in the nursing assistance device 1.

The nursing assistance device 1 stores a record content type for identifying a record content that needs to be recorded for each nursing type.

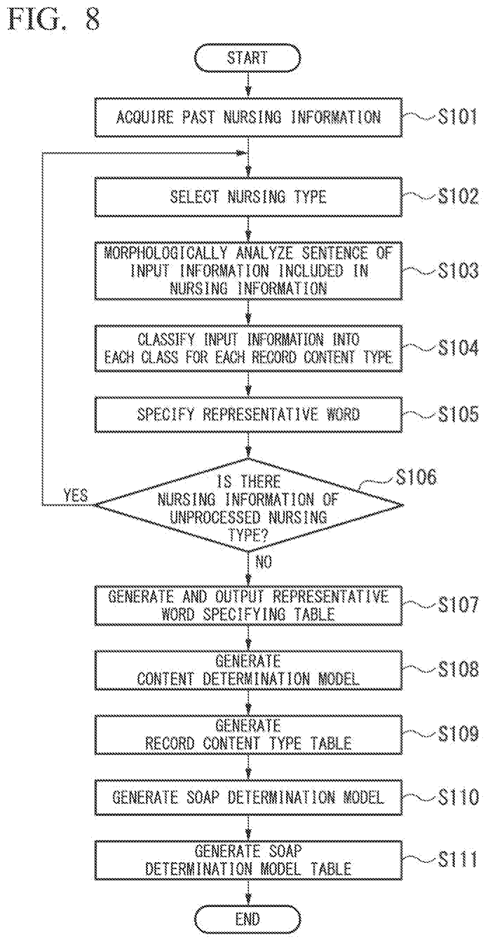

FIG. 8 is a first diagram showing a processing flow of the nursing assistance device 1.

The pre-processing of the nursing assistance device 1 will be described with reference to FIG. 8.

The nursing assistance device 1 acquires pieces of nursing information recorded in an electronic medical record database or the pieces of nursing information stored in the device itself in the past (step S101). The acquired nursing information includes the patient ID, the nurse ID, the nursing type, the SOAP type, the record content type, the input information, and the like. The input information is information input by the user with their voice by using the terminal 2 in the past. The voice from the voice input is converted into characters. The input information converted into the characters is recorded as the nursing information in the electronic medical record database, the database 104, or the like. The input information may be information input as characters on the terminal 2. The model generation unit 12 of the nursing assistance device 1 acquires the pieces of nursing information recorded in the past. For example, in the database 104, words each constituting the input information included in the nursing information in advance and a class of the SOAP type indicated by the input information are given for each nursing type and are included in the nursing information.

The model generation unit 12 extracts the nursing information for each nursing type and selects one nursing type (step S102). The model generation unit 12 performs morphological analysis on the sentence of the input information included in the nursing information of the selected nursing type among all the pieces of nursing information recorded in the database 104 (step S103). The model generation unit 12 classifies (groups) the pieces of input information included in one selected nursing type into classes (groups) for each record content type by using the result of the morphological analysis (step S104). Accordingly, the pieces of input information are classified into several classes such that the pieces of nursing information of which the contents of the pieces input information are similar belong to the same class. The number of classes may be decided in advance or may be decided by using a specific criterion such as an information amount criterion. Here, the class represents the record content type. Hereinafter, the class is referred to as a record content type class. More specifically, as a method of classification (grouping), the model generation unit 12 performs morphological analysis on the input information and generates input information vectors based on the obtained words and the frequencies thereof. The model generation unit 12 classifies (groups) a plurality of pieces of input information into the record content type classes according to the degree of similarity of the vectors. The same record content type class will include a plurality of vectors with high degree of similarity. One vector corresponds to one input information.

The model generation unit 12 specifies a representative word simply representing the contents of the pieces of input information belonging to the record content type class from the words appearing in the pieces of input information included in each record content type class (step S105).

For example, the model generation unit 12 may specify, as the representative word, a word appearing in common in many pieces of input information among the words included in the pieces of input information corresponding to a plurality of vectors belonging to the same record content type class.

Alternatively, the model generation unit 12 may specify, as the representative word, a word that appears in common to many pieces of input information among the words included in the pieces of input information corresponding to the plurality of vectors belonging to the same record content type class and that has a shortest character string length.

The model generation unit 12 determines whether or not there are the pieces of nursing information of the unprocessed nursing type (step S106). In a case where there are the pieces of nursing information of the unprocessed nursing type, the model generation unit 12 selects the next nursing type, and similarly specifies a representative word for each record content type class of the input information in the next nursing type. The user such as the nurse may manually select the input information corresponding to each record content type class, may specify the representative word of each record content type class, and may register the representative word in the nursing assistance device 1.

The model generation unit 12 repeats the same processing until the representative word for each record content type class of the input information in all the pieces of nursing types is specified. Accordingly, the model generation unit 12 specifies “representative word of record content type class C1: walked”, “word of record content type class C2: pain”, and the like based on the nursing information of the nursing type specified by the attributes of the patient that are male, 80s, and fracture. The model generation unit 12 generates a representative word specifying table including a nursing type, a record content type class number, and a representative word, and outputs it to the terminal 2 of the user (step S107). The model generation unit 12 may transmit the representative word specifying table to the predetermined terminal 2.

The model generation unit 12 generates, based on the input information and the record content type class of the input information associated with the input information, the content determination model that determines which record content type class among the plurality of record content type classes in the nursing type designated by the patient ID or the like any input information belongs to (step S108). The content determination model is a model for determining which record content type class the word frequently appears in based on each word obtained from the analysis result of the sentence using the input information. As an example, the content determination model is a model for determining the record content type class in which in a case where a word included in the input information is expressed by any vector, vectors similar to the vector appear unevenly and frequently, based on the vector indicating the word. The model generation unit 12 generates the content determination model table (FIG. 5) representing identification information of the content determination model corresponding to the nursing type, and records it in the database 104.

The user such as the nurse or the doctor may correct the representative word by inputting the word indicating the record content type more appropriately in a representative word input field of the representative word specifying table output on the display of the terminal 2. For example, in a case where the representative word of the record content type class C1 is “walked”, the user corrects the representative word by inputting “walking” as the representative word representing the record content type. In a case where the representative word of the record content type class C2 is “pain”, the user corrects the representative word by inputting a more appropriate “pain degree” as the word representing the record content type. The user instructs the terminal 2 to register the input representative word. The terminal 2 transmits this instruction to the nursing assistance device 1. In response to the instruction, the model generation unit 12 of the nursing assistance device 1 generates the record content type table in which the nursing type, an identification number of the record content type, and the representative word of the record content type are associated with each other, and records it in the database 104 (step S109). Accordingly, for example, as shown in FIG. 7, information of a set of “record content type class C1” and a character string of “walking” as “representative word of record content type” and a set of “record content type class C2” and a character string of “pain degree” as “representative word of record content type” is recorded in the nursing type “fracture”.

The model generation unit 12 generates, based on the input information and the SOAP type class indicated by the input information, the SOAP determination model that determines which class of the types of “Subjective (S)”, “Objective (O)”, “Assessment (A)”, and “Plan (P)” any input information belongs to (step S110). The model generation unit 12 converts the input information included in the newly acquired nursing information into a vector in which a plurality of words included in the input information is represented by directions and sizes in a plurality of dimensions. The model generation unit 12 generates the SOAP determination model that uses, as the input, the vector obtained by converting the input information based on the word constituting the input information included in the acquired nursing information and consequently outputs any type among the SOAP types. The model generation unit 12 generates the SOAP determination model table (FIG. 6) representing identification information of the SOAP determination model corresponding to the nursing type, and records it in the database 104 (step S111).

The pre-processing of the nursing assistance device 1 is ended by the above processing. The content determination model and the SOAP determination model may be retained by the nursing assistance device 1 in advance. The content determination model and the SOAP determination model may be generated by using an algorithm of a general discriminator such as a Support Vector Machine.

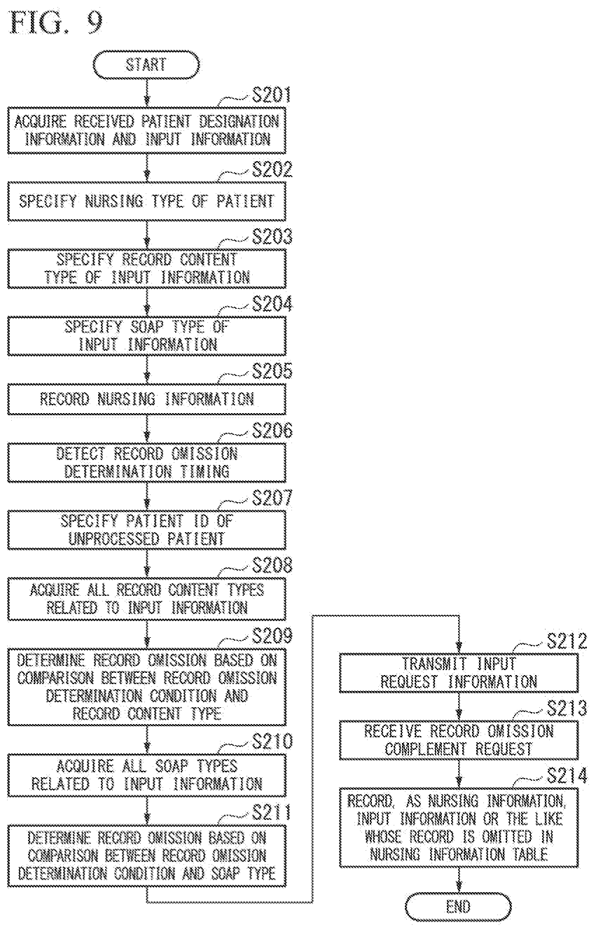

FIG. 9 is a second diagram showing a processing flow of the nursing assistance device 1.

The nursing assistance device 1 performs the input of the input information and the processing of the record omission determination in a state in which the determination model table and the record content type table are generated as described above.

As an example, the user such as the nurse or the doctor activates a nursing assistance application program recorded in the terminal 2. Accordingly, the terminal 2 has functions of outputting a user interface (UI) screen of a nursing assistance business to a display of the terminal 2, inputting the character information by using the UI screen, acquiring the voice input based on an operation on the UI screen, transmitting the input information to the nursing assistance device 1, and outputting various kinds of information received from the nursing assistance device 1 to the UI screen.

The user inputs the information (S) directly obtained from the patient, the information (O) obtained from the physical examination and the inspection by the nurse, the information (A) of the assessment of the patient, and the information (P) indicating the performed plan or treatment by using the terminal 2 at an appropriate timing The input may be performed by voice, or may be input by character input using the input function provided in the terminal 2. At this time, the user designates necessary patient designation information such as the patient ID and the nurse ID by using the UI screen. Alternatively, the necessary patient designation information such as the patient ID and the nurse ID may be input by voice. The terminal 2 transmits the patient designation information and the input information to the nursing assistance device 1.

The acquisition unit 13 acquires the input information and the patient designation information received by the communication module 105 (reception unit) of the nursing assistance device 1 (step S201). The acquisition unit 13 outputs the input information to the type determination unit 14. The type determination unit 14 specifies the nursing type of the patient recorded in the database in advance in association with the patient ID included in the patient designation information (step S202). This nursing type is determined according to the patient attributes such as a gender, an age, and a disease of the patient. The type determination unit 14 specifies the content determination model based on the information recorded in the content determination model table and the nursing type of the patient. The type determination unit 14 acquires a model formula of the content determination model from a storage unit or the like. The type determination unit 14 performs morphological analysis on the input information, and inputs the input information vector generated by the obtained words and the frequencies thereof into the content determination model. As a result of the input, the type determination unit 14 specifies the record content type of the input information by the determination processing using the content determination model (step S203).

The type determination unit 14 also specifies the SOAP determination model based on the information recorded in the content determination model table and the nursing type of the patient. The type determination unit 14 acquires a model formula of the SOAP determination model from the storage unit or the like. The type determination unit 14 performs morphological analysis on the input information, and inputs the input information vector generated by the obtained words and the frequencies thereof into the SOAP determination model. As a result of the input, the type determination unit 14 specifies the SOAP type of the input information by the determination processing using the SOAP determination model (step S204).

The acquisition unit 13 outputs the patient designation information, the input information, the nursing type, and the record content type to the recording unit 15. The recording unit 15 records the nursing information (step S205). The recording unit 15 writes the patient ID, the nurse ID, the nursing type, the SOAP type, the record content type, and the input information included in a set type in association with each other in the nursing information table.

The acquisition unit 13 and the type determination unit 14 repeat the above processing whenever the nursing assistance device 1 receives the input information received from the terminal 2. Accordingly, the input information of the patient is accumulated as the nursing information in the nursing information table. The record omission determination unit 16 determines the record omission as the nursing information from the pieces of input information accumulated by recording the nursing information at a predetermined timing. Specifically, the record omission determination unit 16 detects a predetermined record omission determination timing such as one hour before a set switching time of a work shift of the nurse (step S206). The record omission determination unit 16 sorts the pieces of unprocessed nursing information of the omission determination accumulated at this timing by the patient ID. The record omission determination unit 16 specifies the patient ID of the first unprocessed patient (step S207).

The record omission determination unit 16 acquires all the record content types related to the pieces of input information recorded in the database 104 in association with the patient ID (step S208). The record omission determination unit 16 acquires the record omission determination condition. This record omission determination condition indicates a specific record type among a plurality of different record types, and is recorded in advance for each patient by the user such as the nurse or the doctor. As an example, the record omission determination unit 16 determines the record omission based on the comparison between the record omission determination condition specific to the patient and the record content types included in the nursing information of the patient indicated by the patient ID (step S209). As a specific example, a case where the record omission determination condition indicates that input information indicating a predetermined record content type is required as the nursing information will be described. That is, in this case, the record content type for specifying the input information required as the nursing information is included in the record omission determination condition. The record omission determination unit 16 determines whether the record type included in the record omission determination condition matches any record content type acquired from the nursing information table based on the patient ID. In a case where the record type included in the record omission determination condition does not match any one of the record content types acquired from the nursing information table based on the patient ID, the record omission determination unit 16 determines that the record omission occurs. That is, in a case where the record type included in the record omission determination condition does not match any of the record content types acquired from the nursing information table based on the patient ID, the record omission determination unit 16 determines that the record omission occurs.

As another specific example of the processing of determining the record omission, a case where the record omission determination condition indicates that the input information including a predetermined keyword needs to be recorded as the nursing information will be described. That is, in this case, a keyword for specifying the input information required as the nursing information is included in the omission determination condition. The record omission determination unit 16 determines whether the keyword included in the record omission determination condition matches any word included in the pieces of input information in the nursing information acquired from the nursing information table based on the patient ID. In a case where the keyword included in the record omission determination condition does not match any word included in the pieces of input information in the nursing information acquired from the nursing information table based on the patient ID, the record omission determination unit 16 determines that the record omission occurs.

The record omission determination unit 16 acquires all the SOAP types related to the pieces of input information recorded in the database 104 in association with the patient ID (step S210). The record omission determination unit 16 acquires the record omission determination condition. As described above, this record omission determination condition indicates a specific record type among the plurality of different record types such as the record content type and the SOAP type, and is recorded in advance for each patient by the user such as the nurse or the doctor. As another example, the record omission determination unit 16 determines the record omission based on the comparison between the record omission determination condition specific to the patient and the SOAP type and the record content type included in the nursing information of the patient indicated by the patient ID (step S211). In this example, the record omission determination condition indicates that input information indicating a predetermined SOAP type and a predetermined record content type is required as nursing information. That is, in this case, the SOAP type and the record content type for specifying the input information required as the nursing information are included in the record omission determination condition. The record omission determination unit 16 determines whether there is the record omission determination condition matching the record type (combination of SOAP type and record content type) included in the record omission determination condition among the combinations of the SOAP type and the record content type acquired from the nursing information table based on the patient ID. In a case where the record type included in the record omission determination condition does not match any of the combinations of the SOAP types and the record content types acquired from the nursing information table based on the patient ID, the record omission determination unit 16 determines that the record omission occurs.

The record omission determination unit 16 acquires the record content type and the SOAP type of the target determined to be record omission and the patient ID. The record omission determination unit 16 acquires a nurse ID indicating each nurse of a nursing group that nurses the patient indicated by the ID or a nurse ID indicating a specific nurse based on the patient ID. The record omission determination unit 16 may acquire the patient attributes (name, gender, room number, and bed number) and the like recorded in the database 104 in association with the patient ID. The record omission determination unit 16 outputs the acquired information to the output unit 17. The output unit 17 generates input request information for the record omission including the nurse ID, the patient ID, the SOAP type, the record content type, and the patient attribute. The output unit 17 transmits the input request information to the terminal 2 owned by the nurse with the ID based on the nurse ID (step S212). The terminal 2 specified based on the nurse ID receives the input request information.

The terminal 2 receives the input request information. The terminal 2 displays a UI screen showing input fields of the patient ID, the SOAP type, the record content type, the patient attributes, the input information, and the like included in the input request information on the display. The nurse specifies the patient based on the patient ID and the patient attributes displayed on the display of the terminal 2. The nurse inputs the input information regarding the SOAP type and the record content type displayed on the display. For example, it is assumed that the patient is a fracture patient, the SOAP type indicates the information (A) of the assessment of the patient, and the record content type indicates “walking”. In this case, the nurse inputs, as the input information, the information of the assessment of walking for the specified patient. The nurse may input the input information by voice or by characters. In a case where it is input by voice, a microphone function of the terminal 2 receives the voice and generates voice data. The terminal 2 transmits a record omission complement request including: the patient ID, the nursing ID, the SOAP type, and the record content type included in the input request information; and the input information to the nursing assistance device 1 based on the instruction of the user such as the nurse.

The nursing assistance device 1 receives the record omission complement request (step S213). The recording unit 15 of the nursing assistance device 1 records, as the nursing information, the input information or the like whose record is omitted in the nursing information table based on the information included in the record omission complement request (step S214). At a timing such as one hour before the switching time of the work shift of the nurse, the nurse in charge of the patient until this point in time is notified of the record omission. The information whose record is omitted is input in response to the notification, and thus, appropriate nursing information for the patient is handed over to the nurse in charge of the same patient in the next work shift. Accordingly, the labor of the nurse can be reduced. In other words, in a case where the nurse in charge of the same patient in the next work shift notices the information whose record is omitted and the information is important for nursing the patient, the nurse separately asks the nurse of the previous work shift. However, according to the example embodiment of the present invention, since the information whose record is omitted is input, it is not necessary for the nurse in charge of the same patient in the next work shift to separately ask the nurse in the previous work shift.

According to the above-mentioned processing, since the user such as the nurse is specifically notified of the SOAP type whose record is omitted for the patient and the record content type specified based on the record omission determination condition, the user such as the nurse can be notified of a required minimum record omission indicated by the record omission determination condition. Accordingly, unnecessary information can be prevented from being registered, and a work volume of the nurse can be reduced. Moreover, the nurse can appropriately leave, as information, the nursing information of the patient.

In the above-mentioned processing, the record omission determination unit 16 determines the record omission in the nursing information by using the record omission determination condition. However, the record omission determination unit 16 may determine the nursing information whose record is omitted based on the nursing information of the patient and a biological change of the patient. For example, when there is no change in a body temperature, it is not particularly necessary to record the nursing information for the body temperature. Accordingly, the record omission determination unit 16 may perform processing such as not performing the record omission determination for the body temperature in a case where there is no change in the body temperature based on the record omission determination condition in which there is no change in the body temperature. For example, in a case where there is a change in the body temperature, it is necessary to record the nursing information for the body temperature. Accordingly, the record omission determination unit 16 may perform processing such as performing the record omission determination for the body temperature in a case where there is a change in the body temperature based on the record omission determination condition in which there is the change in the body temperature.

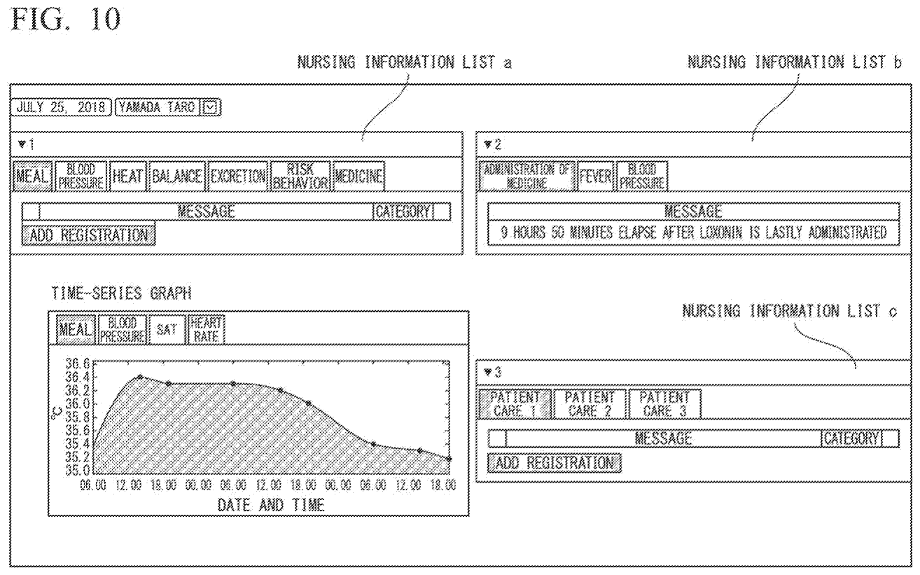

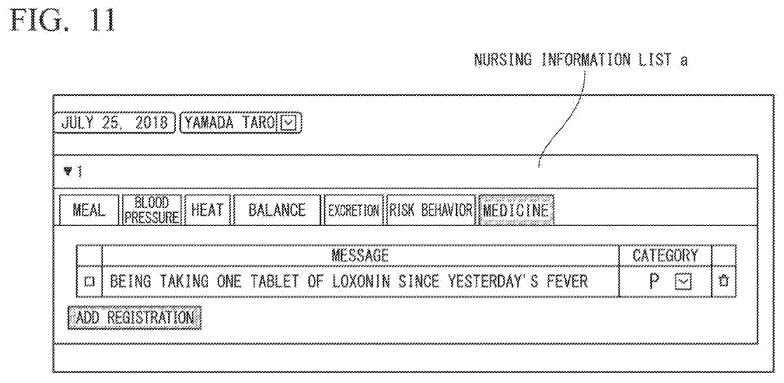

FIG. 10 is a diagram showing a first example of the UI screen.

The terminal 2 is communicably connected to the nursing assistance device 1, and transmits the nursing information request based on a user operation to output a UI screen including a nursing information list to the display. The terminal 2 displays a UI screen including a nursing information list a, a nursing information list b, and a nursing information list c of the patient having the patient ID specified by the user operation. The terminal 2 generates, based on biological information such as a body temperature or a blood pressure included in the nursing information, its transition graph and displays it on the UI screen.

FIG. 11 is a diagram showing a second example of the UI screen.

FIG. 11 is an enlarged view of the nursing information list a in the UI screen shown in FIG. 10. As an example, in the nursing information list a, the input information and the SOAP type (category) indicated by the input information are displayed for each record content type such as heat, walking balance, excretion, and administered medicine for a meal and a blood pressure.

The nurse can display such a UI screen on the terminal 2 and confirm a past nursing history of the patient.

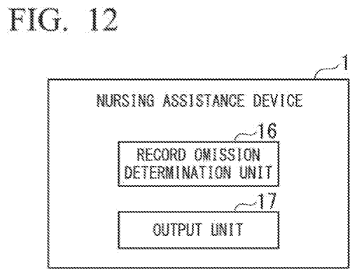

FIG. 12 is a diagram showing a nursing assistance device 1 according to another example embodiment.

As shown in FIG. 12, the nursing assistance device 1 includes at least a record omission determination unit 16 and an output unit 17.

The record omission determination unit 16 determines the nursing information whose record is omitted for the patient among the pieces of nursing information of the patient based on the record omission determination condition of the nursing information.

The output unit 17 outputs an input request for the nursing information whose record is omitted.

The above-mentioned nursing assistance device 1 has a computer system inside. A procedure of the processing described above is stored in a computer-readable recording medium in the form of a program, and the processing is performed by the computer reading and executing this program.

The program may be for realizing a part of the above-mentioned functions. The program may be a so-called difference file (difference program) that can realize the above-mentioned functions in combination with a program already recorded in the computer system.

This application is based upon and claims the benefit of priority from Japanese patent application No. 2018-199529, filed Oct. 23, 2018, the disclosure of which is incorporated herein in its entirety by reference.

### INDUSTRIAL APPLICABILITY

The present invention may be applied to a nursing assistance device, a nursing assistance method, and a recording medium.

### REFERENCE SYMBOLS

1: Nursing assistance device

2: Terminal

11: Control unit

12: Model generation unit

13: Acquisition unit

14: Type determination unit

15: Recording unit

16: Record omission determination unit

17: Output unit

100: Nursing assistance system

What is claimed is:

1. A nursing assistance device comprising:
at least one memory configured to store instructions; and
at least one processor configured to execute the instructions to:
determine information whose record is omitted among nursing information related to nursing of a patient, based on a record omission determination condition related to the nursing information; and
output an input request for the determined information whose recording is omitted.

2. The nursing assistance device according to claim 1,
wherein the at least one processor is configured to execute the instructions to:
determine at least a record type of input information; and
record, as the nursing information, the input information associated with the record type.

3. The nursing assistance device according to claim 2, wherein determining the information comprises determining the information whose record is omitted based on comparison between a specified record type indicated by the omission determination condition and the record type included in the nursing information.

4. The nursing assistance device according to claim 2, wherein determining the information comprises determining the information whose record is omitted based on the nursing information of the patient and a biological change of the patient.

5. The nursing assistance device according to claim 1,
wherein the at least one processor is configured to execute the instructions to: receive input information from a terminal.

6. The nursing assistance device according to claim 2,
wherein the at least one processor is configured to execute the instructions to:
generate, for each nursing type, a determination model for specifying a record type based on a plurality of pieces of nursing information of patients whose nursing types match each other;
specify a determination model corresponding to a nursing type of the patient from the determination model for each the nursing type; and
input information to the specified determination model, and
determining the at least the record type of the input information comprises determining the record type of the input information based on an output from the specified determination model.

7. A nursing assistance method comprising:
determining information whose record is omitted among nursing information related to nursing of a patient, based on a record omission determination condition related to the nursing information; and
outputting an input request for the determined information whose recording is omitted.

8. A non-transitory recording medium storing a program for causing a computer to execute:
determining information whose record is omitted among nursing information related to nursing of a patient, based on a record omission determination condition related to the nursing information; and
outputting an input request for the determined information whose recording is omitted.
